(19) United States
(12) Patent Application Publication
(10) Pub. No.: US 2011/0251147 A1
(43) Pub. Date: Oct. 13, 2011

(54) Compositions Comprising Quinolone and Methods for Treating or Controlling Infections

(76) Inventors: Srini Venkatesh, Pittsford, NY (US); Hongna Wang, Fairport, NY (US); Erning Xia, Penfield, NY (US); Matthew S. Jonasse, Webster, NY (US)

(21) Appl. No.: 13/167,836

(22) Filed: Jun. 24, 2011

Related U.S. Application Data
(62) Division of application No. 12/205,946, filed on Sep. 8, 2008.
(60) Provisional application No. 60/974,141, filed on Sep. 21, 2007.

(51) Int. Cl. A61K 31/55; A61K 31/7036; A61P 31/12; A61P 31/04; A61P 31/10; A61K 31/7076; A61K 31/7048; A61K 31/551

(52) U.S. Cl. 514/31; 514/39; 514/217.07; 514/218; 514/45

(57) ABSTRACT

Compositions for treating or controlling infections comprise a fluoroquinolone having Formulae I-VIII and have a pH in the range from about 3.5 to about 5.5 or from about 10.5 to about 12. Methods for treating or controlling such infection use such compositions. Such compositions and methods can deliver a higher amount of the fluoroquinolone to the affected site.

### CROSS-REFERENCE

This application is a divisional of application Ser. No. 12/205,946 filed Sep. 8, 2011 and claims the benefit of Provisional Patent Application No. 60/974,141 filed Sep. 21, 2007 which are both incorporated by reference herein.

### BACKGROUND OF THE INVENTION

The present invention relates to compositions and methods for treating or controlling infections. In particular, the present invention relates to such compositions comprising a quinolone carboxylic acid or a derivative thereof, and to methods of using such compositions.

Pathogens continue to pose a serious threat to public health as indicated by a worldwide resurgence of diseases caused by bacteria, fungi, and/or viruses. Infections by pathogenic microorganisms affect a large number of patients every year. Common infections include those of the eye, ear, and respiratory system. An experienced medical practitioner often can determine the etiology of an infection and, therefore, prescribe an effective treatment. However, infections are often caused by mixed types of microorganisms that may not be immediately obvious on presentation. Consequently, an initial treatment regimen may not be immediately effective and must be replaced with another. In cases where the patients are especially vulnerable, such as those with a compromised immune system, a delayed onset of a beneficial effect of the treatment may lead to increased risk of more serious complications.

Otitis media, an infection of the middle ear, is a major worldwide infection in children. By the age of 2 years, seventy percent of children have experienced at least one episode of acute otitis media (“AOM”). T. Heikkinen et al., Clin. Microbiol. Rev., Vol. 16, No. 2, 230 (2003). Otitis media can also occur in adults. AOM is generally considered a bacterial infection that is treated with antibiotics. The three most common bacteria isolated from the middle ear fluid (“MEF”) are Streptococcus pneumoniae, Haemophilus influenzae, and Moraxella catarrhalis. See; e.g., J. T. Kirchener, American Family Physician, Apr. 15, 1999. In cases of otitis externa, the most common bacteria are Pseudomonas aeruginosa and Staphylococcus aureus. Fungi have also been found to a lesser extent in cases of otic infections.

Infections of the upper respiratory system are also common. The common cold is mostly of viral etiology. However, bacteria and fungi have also often caused other infections of the upper respiratory system. Bacteria are the most common infectious agents in sinusitis. Streptococcus pneumoniae, Haemophilus influenzae, and Moraxella catarrhalis have been found in most of cases of sinusitis. Other possible bacterial culprits include other streptococcal strains and Staphylococcus aureus.

Keratitis and conjunctivitis, two common ocular infections, are caused mostly by bacteria, fungi, and/or viruses. Among these microorganisms, numerous cocci (Staphylococcus, Streptococcus, and Neisseria species) and bacilli (Corynebacterium, Propionobacterium, Clostridium, Pseudomonas, Klebsiella, Hemophilus, Moraxella, Proteus, Serratia, Escherichia, and Enterobacter species) have been isolated from cases of ocular infections.

Quinolones comprise a family of newer antibacterial agents, some of which have been used for the foregoing conditions. However, quinolones typically have low solubility, and thus, extra measures have been used to increase their availability at the target sites.

An additional challenge in the treatment of infections is the emergence of bacterial resistance to antibiotics. Such resistance may be attributed to prior widespread, and largely effective, therapeutic and prophylactic use of antibiotics, which, unfortunately, over time has also selected for resistant strains of various bacterial pathogens. Of particular concern to the public health have been the emergence and proliferation of bacterial strains that are resistant to multiple antibiotics in the current arsenal of antimicrobial agents. Therefore, a condition may not respond to an initially prescribed therapy, and, in such a case, another medicament must be given, resulting in delayed control of the pathogen.

Therefore, there is a continued need to develop improved pharmaceutical compositions for combating infections. It is also very desirable to provide pharmaceutical compositions that can overcome at least some of the shortcomings of prior-art antimicrobial agents.

### SUMMARY OF THE INVENTION

In general, the present invention provides pharmaceutical compositions and methods using such compositions for the treatment or control of infections.

In one aspect, the present invention provides pharmaceutical compositions and methods using such compositions for the treatment or control of infections of an eye, an ear, a portion of a respiratory system, or a combination thereof.

In another aspect, a pharmaceutical composition comprises a high concentration of a quinolone soluble in a pharmaceutically acceptable vehicle.

In still another aspect, the pharmaceutical composition comprises an aqueous solution.

In yet another aspect, the pharmaceutical composition is effective against at least a bacterium that is resistant to at least a prior-art antibacterial agent.

In a further aspect, such quinolone comprises at least one member of a family of fluoroquinolones that have Formula I or salts thereof,

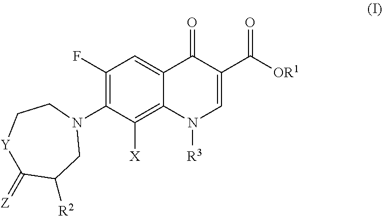

wherein R¹ is selected from the group consisting of hydrogen, unsubstituted lower alkyl groups, substituted lower alkyl groups, cycloalkyl groups, unsubstituted C₅-C₂₄ aryl groups, substituted C₅-C₂₄ aryl groups, unsubstituted C₅-C₂₄ heteroaryl groups, substituted C₅-C₂₄ heteroaryl groups, and groups that can be hydrolyzed in living bodies; R² is selected from the group consisting of hydrogen, unsubstituted amino group, and amino groups substituted with one or two lower alkyl groups; R³ is selected from the group consisting of hydrogen, unsubstituted lower alkyl groups, substituted lower alkyl groups, cycloalkyl groups, unsubstituted lower alkoxy groups, substituted lower alkoxy groups, unsubstituted C₅-C₂₄ aryl groups, substituted C₅-C₂₄ aryl groups, unsubstituted C₅-C₂₄ heteroaryl groups, substituted C₅-C₂₄ heteroaryl groups, unsubstituted C₅-C₂₄ aryloxy groups, substituted C₅-C₂₄ aryloxy groups, unsubstituted C₅-C₂₄ heteroaryloxy groups, substituted C₅-C₂₄ heteroaryloxy groups, and groups that can be hydrolyzed in living bodies; X is selected from the group consisting of halogen atoms; Y is selected from the group consisting of CH₂, O, S, SO, SO₂, and NR⁴, wherein R⁴ is selected from the group consisting of hydrogen, unsubstituted lower alkyl groups, substituted lower alkyl groups, and cycloalkyl groups; and Z is selected from the group consisting of oxygen and two hydrogen atoms; and wherein the compositions are capable of inhibiting a growth or survival of mixed types of microorganisms causing said infections.

In still another aspect, said quinolone comprises a member of a family of fluoroquinolones having Formula II or salts thereof,

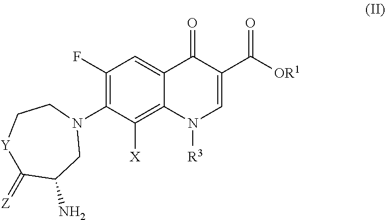

wherein R¹, R³, X, Y, and Z have the meanings as disclosed above; and wherein the composition is capable of inhibiting a growth or survival of mixed types of microorganisms causing an infection.

In still another aspect, the present invention provides a method for treating or controlling an infection of an eye, an ear, a portion of a respiratory system, or a combination thereof. The method comprises administering a composition comprising a fluoroquinolone having Formula I or II soluble therein at a high concentration to a site of infection to treat or control said infection.

In one embodiment, the method comprises topically administering such a composition. In another embodiment, the method comprises orally administering such a composition.

In yet another aspect, said infection comprises infections of an eye, an ear, a portion of an upper respiratory system, or a combination thereof.

Other features and advantages of the present invention will become apparent from the following detailed description and claims and the appended figures.

### BRIEF DESCRIPTION OF THE FIGURE

FIG. 1 shows the solubility of the compound having Formula IV in USP water.

### DETAILED DESCRIPTION

As used herein, the term “control” includes alleviation, reduction, amelioration, and prevention.

As used herein, the term “lower alkyl” or “lower alkyl group” means a C₁-C₁₅ linear- or branched-chain saturated aliphatic hydrocarbon monovalent group, which may be unsubstituted or substituted. The group may be partially or completely substituted with halogen atoms (F, Cl, Br, or I). Non-limiting examples of lower alkyl groups include methyl, ethyl, n-propyl, 1-methylethyl (or isopropyl), n-butyl, n-pentyl, 1,1-dimethylethyl (or t-butyl), and the like. It may be abbreviated as “Alk”.

As used herein, the term “lower alkoxy” or “lower alkoxy group” means a C₁-C₁₅ linear- or branched-chain saturated aliphatic alkoxy monovalent group, which may be unsubstituted or substituted. The group may be partially or completely substituted with halogen atoms (F, Cl, Br, or I). Non-limiting examples of lower alkoxy groups include methoxy, ethoxy, n-propoxy, 1-methylethoxy (isopropoxy), n-butoxy, n-pentoxy, t-butoxy, and the like.

The term “cycloalkyl” or “cycloalkyl group” means a stable aliphatic saturated 3- to 15-membered monocyclic or polycyclic monovalent radical consisting solely of carbon and hydrogen atoms which may comprise one or more fused or bridged ring(s), preferably a 3- to 7-membered monocyclic rings. Other exemplary embodiments of cycloalkyl groups include 7- to 10-membered bicyclic rings. Unless otherwise specified, the cycloalkyl ring may be attached at any carbon atom which results in a stable structure and, if substituted, may be substituted at any suitable carbon atom which results in a stable structure. Exemplary cycloalkyl groups include cyclopropyl, cyclobutyl, cyclopentyl, cyclohexyl, cycloheptyl, cyclooctyl, cyclononyl, cyclodecyl, norbornyl, adamantyl, tetrahydronaphthyl (tetralin), 1-decalinyl, bicyclo[2.2.2]octanyl, 1-methylcyclopropyl, 2-methylcyclopentyl, 2-methylcyclooctyl, and the like.

As used herein, the term “aryl” or “aryl group” means an aromatic carbocyclic monovalent or divalent radical. In some embodiments, the aryl group has a number of carbon atoms from 5 to 24 and has a single ring (e.g., phenyl or phenylene), multiple condensed rings (e.g., naphthyl or anthranyl), or multiple bridged rings (e.g., biphenyl). Unless otherwise specified, the aryl ring may be attached at any suitable carbon atom which results in a stable structure and, if substituted, may be substituted at any suitable carbon atom which results in a stable structure. Non-limiting examples of aryl groups include phenyl, naphthyl, anthryl, phenanthryl, indanyl, indenyl, biphenyl, and the like. It may be abbreviated as “Ar”.

The term “heteroaryl” or “heteroaryl group” means a stable aromatic monocyclic or polycyclic monovalent or divalent radical, which may comprise one or more fused or bridged ring(s). In some embodiments, the heteroaryl group has 5-24 members, preferably a 5- to 7-membered monocyclic or 7- to 10-membered bicyclic radical. The heteroaryl group can have from one to four heteroatoms in the ring(s) independently selected from nitrogen, oxygen, and sulfur, wherein any sulfur heteroatoms may optionally be oxidized and any nitrogen heteroatom may optionally be oxidized or be quaternized. Unless otherwise specified, the heteroaryl ring may be attached at any suitable heteroatom or carbon atom which results in a stable structure and, if substituted, may be substituted at any suitable heteroatom or carbon atom which results in a stable structure. Non-limiting examples of heteroaryls include furanyl, thienyl, pyrrolyl, oxazolyl, thiazolyl, imidazolyl, pyrazolyl, isoxazolyl, isothiazolyl, oxadiazolyl, triazolyl, tetrazolyl, thiadiazolyl, pyridinyl, pyridazinyl, pyrimidinyl, pyrazinyl, triazinyl, indolizinyl, azaindolizinyl, indolyl, azaindolyl, diazaindolyl, dihydroindolyl, dihydroazaindoyl, isoindolyl, azaisoindolyl, benzofuranyl, furanopyridinyl, furanopyrimidinyl, furanopyrazinyl, furanopyridazinyl, dihydrobenzofuranyl, dihydrofuranopyridinyl, dihydrofuranopyrimidinyl, benzothienyl, thienopyridinyl, thienopyrimidinyl, thienopyrazinyl, thienopyridazinyl, dihydrobenzothienyl, dihydrothienopyridinyl, dihydrothienopyrimidinyl, indazolyl, azaindazolyl, diazaindazolyl, benzimidazolyl, imidazopyridinyl, benzthiazolyl, thiazolopyridinyl, thiazolopyrimidinyl, benzoxazolyl, benzoxazinyl, benzoxazinonyl, oxazolopyridinyl, oxazolopyrimidinyl, benzisoxazolyl, purinyl, chromanyl, azachromanyl, quinolizinyl, quinolinyl, dihydroquinolinyl, tetrahydroquinolinyl, isoquinolinyl, dihydroisoquinolinyl, tetrahydroisoquinolinyl, cinnolinyl, azacinnolinyl, phthalazinyl, azaphthalazinyl, quinazolinyl, azaquinazolinyl, quinoxalinyl, azaquinoxalinyl, naphthyridinyl, dihydronaphthyridinyl, tetrahydronaphthyridinyl, pteridinyl, carbazolyl, acridinyl, phenazinyl, phenothiazinyl, and phenoxazinyl, and the like.

In general, the present invention provides a pharmaceutical composition and a method for treating or controlling an infection.

In one aspect, the present invention provides pharmaceutical compositions and methods using such compositions for the treatment or control of infections of an eye, an ear, a portion of a respiratory system, or a combination thereof.

In another aspect, a pharmaceutical composition comprises a high concentration of a quinolone soluble in a pharmaceutically acceptable vehicle.

In a still another aspect, said concentration is greater than about 0.05 percent by weight (or, alternatively, greater than about 0.1, or 1 percent by weight) of the total composition.

In still another aspect, said concentration is in the range from about 0.05 percent to about 2 percent by weight (or, alternatively, from about 0.05 percent to about 1 percent by weight) of the total composition.

In yet another aspect, the pharmaceutical composition comprises an aqueous solution.

In yet another aspect, the pharmaceutical composition is effective against at least a bacterium that is resistant to at least a prior-art antibacterial agent.

In a further aspect, such quinolone comprises at least one member of a family of fluoroquinolones that have Formula I or salts thereof,

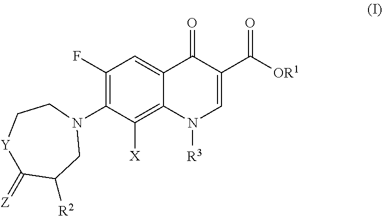

wherein R¹ is selected from the group consisting of hydrogen, unsubstituted lower alkyl groups, substituted lower alkyl groups, cycloalkyl groups, unsubstituted C₅-C₂₄ aryl groups, substituted C₅-C₂₄ aryl groups, unsubstituted C₅-C₂₄ heteroaryl groups, substituted C₅-C₂₄ heteroaryl groups, and groups that can be hydrolyzed in living bodies; R² is selected from the group consisting of hydrogen, unsubstituted amino group, and amino groups substituted with one or two lower alkyl groups; R³ is selected from the group consisting of hydrogen, unsubstituted lower alkyl groups, substituted lower alkyl groups, cycloalkyl groups, unsubstituted lower alkoxy groups, substituted lower alkoxy groups, unsubstituted C₅-C₂₄ aryl groups, substituted C₅-C₂₄ aryl groups, unsubstituted C₅-C₂₄ heteroaryl groups, substituted C₅-C₂₄ heteroaryl groups, unsubstituted C₅-C₂₄ aryloxy groups, substituted C₅-C₂₄ aryloxy groups, unsubstituted C₅-C₂₄ heteroaryloxy groups, substituted C₅-C₂₄ heteroaryloxy groups, and groups that can be hydrolyzed in living bodies; X is selected from the group consisting of halogen atoms; Y is selected from the group consisting of CH₂, O, S, SO, SO₂, and NR⁴, wherein R⁴ is selected from the group consisting of hydrogen, unsubstituted lower alkyl groups, substituted lower alkyl groups, and cycloalkyl groups; and Z is selected from the group consisting of oxygen and two hydrogen atoms; and wherein the compositions are capable of inhibiting a growth or survival of mixed types of microorganisms causing said infections.

In another aspect, said infection is caused by mixed types of bacteria.

In one aspect, said infection is selected from the group consisting of otitis, sinusitis, nasophrayngitis, orophrayngitis, epiglottitis, laryngotracheitis, bronchitis, bronchiolitis, pneumonia, keratitis, conjunctivitis, blepharitis, hordeolum, phlyctenulosis, endophthalmitis, preseptal and orbital cellulites, dacryocystitis, and combinations thereof.

In one aspect, R¹ is selected from the group consisting of hydrogen, C₁-C₅ (or alternatively, C₁-C₃) substituted and unsubstituted alkyl groups, C₃-C₁₀ (or alternatively, C₃-C₅) cycloalkyl groups, C₅-C₁₄ (or alternatively, C₆-C₁₄, or C₅-C₁₀, or C₆-C₁₀) substituted and unsubstituted aryl groups, C₅-C₁₄ (or alternatively, C₆-C₁₄, or C₅-C₁₀, or C₆-C₁₀) substituted and unsubstituted heteroaryl groups, and groups that can be hydrolyzed in living bodies. In one embodiment, R¹ is selected from the group consisting of C₁-C₅ (or alternatively, C₁-C₃) substituted and unsubstituted alkyl groups.

In another aspect, R² is selected from the group consisting of unsubstituted amino group and amino groups substituted with one or two C₁-C₅ (or alternatively, C₁-C₃) alkyl groups.

In still another aspect, R³ is selected from the group consisting of hydrogen, C₁-C₅ (or alternatively, C₁-C₃) substituted and unsubstituted alkyl groups, C₃-C₁₀ (or alternatively, C₃-C₅) cycloalkyl groups, C₁-C₅ (or alternatively, C₁-C₃) substituted and unsubstituted alkoxy groups, C₅-C₁₄ (or alternatively, C₆-C₁₄, or C₅-C₁₀, or C₆-C₁₀) substituted and unsubstituted aryl groups, C₅-C₁₄ (or alternatively, C₆-C₁₄, or C₅-C₁₀, or C₆-C₁₀) substituted and unsubstituted heteroaryl groups, and C₅-C₁₄ (or alternatively, C₆-C₁₄, or C₅-C₁₀, or C₆-C₁₀) substituted and unsubstituted aryloxy groups. In one embodiment, R³ is selected from the group consisting of C₃-C₁₀ (or alternatively, C₃-C₅) cycloalkyl groups.

In yet another aspect, X is selected from the group consisting of Cl, F, and Br. In one embodiment, X is Cl. In another embodiment, X is F.

In a further aspect, Y is CH₂ and Z comprises two hydrogen atoms.

In still another aspect, Y is NH, Z is O, and X is Cl.

Some non-limiting members of the family of compounds having Formula I are shown in Table I. Other compounds of the family not listed in Table 1 are also suitable in selected situations.

TABLE 1
Some Selected Fluoroquinolones

| Com- | | | | | | |
|---|---|---|---|---|---|---|
| pound | R¹ | R² | R³ | X | Y | Z |
| 1 | H | H | CH₃ | Cl | CH₂ | 2 H |
| 2 | H | NH₂ | CH₃ | Cl | CH₂ | 2 H |
| 3 | H | NH₂ | cyclopropyl | Cl | CH₂ | 2 H |
| 4 | H | NH(CH₃) | cyclopropyl | Cl | CH₂ | 2 H |
| 5 | H | N(CH₃)₂ | cyclopropyl | Cl | CH₂ | 2 H |
| 6 | CH₃ | NH₂ | cyclopropyl | Cl | CH₂ | 2 H |
| 7 | C₂H₅ | NH₂ | cyclopropyl | Cl | CH₂ | 2 H |
| 8 | H | NH₂ | cyclopropyl | F | CH₂ | 2 H |
| 9 | H | NH₂ | cyclopropyl | Br | CH₂ | 2 H |
| 10 | H | NH(C₂H₅) | cyclopropyl | Cl | CH₂ | 2 H |
| 11 | H | NH(C₂H₅) | cyclopropyl | F | CH₂ | 2 H |
| 12 | H | NH₂ | cyclopentyl | Cl | CH₂ | 2 H |
| 13 | H | NH₂ | cyclopropyl | Cl | CH₂ | O |
| 14 | H | NH₂ | cyclopropyl | F | CH₂ | O |
| 15 | H | NH₂ | cyclopropyl | Br | CH₂ | O |
| 16 | H | NH₂ | cyclopropyl | Cl | CH(C₃H₅) | O |
| 17 | CH₃ | NH₂ | cyclopropyl | Cl | CH₂ | O |
| 18 | CH₃ | NH(CH₃) | cyclopropyl | Cl | CH₂ | O |
| 19 | CH₃ | N(CH₃)₂ | cyclopropyl | Cl | CH₂ | O |
| 20 | CH₃ | NH(C₂H₅) | cyclopropyl | Cl | CH₂ | O |
| 21 | CH₃ | NH(C₃H₇) | cyclopropyl | Cl | CH₂ | O |
| 22 | CH₃ | N(CH₃)(C₂H₅) | cyclopropyl | Cl | CH₂ | O |
| 23 | H | NH₂ | cyclopropyl | Cl | NH | O |
| 24 | CH₃ | NH(CH₃) | cyclopropyl | Cl | NH | 0 |
| 25 | H | 2H | cyclopropyl | Cl | NH | O |

In one embodiment, the fluoroquinolone carboxylic acid included in a composition of the present invention has Formula III.

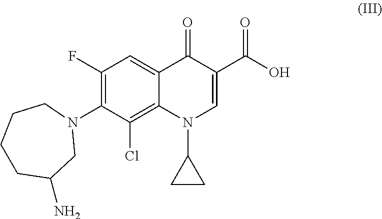

In another embodiment, the fluoroquinolone carboxylic acid included in a composition of the present invention has Formula IV, V, or VI.

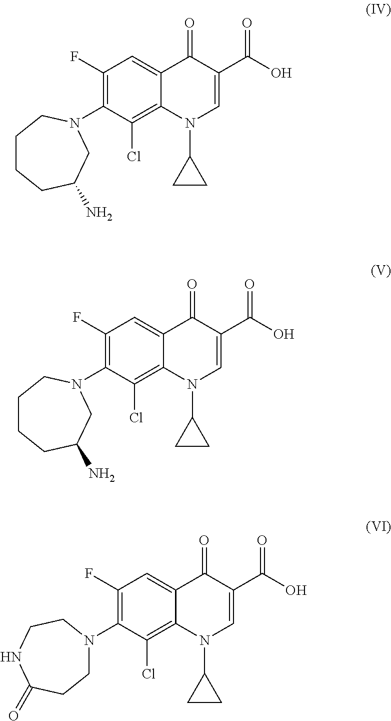

In still other embodiments, the fluoroquinolone carboxylic acid included in a composition of the present invention has Formula VII or VIII.

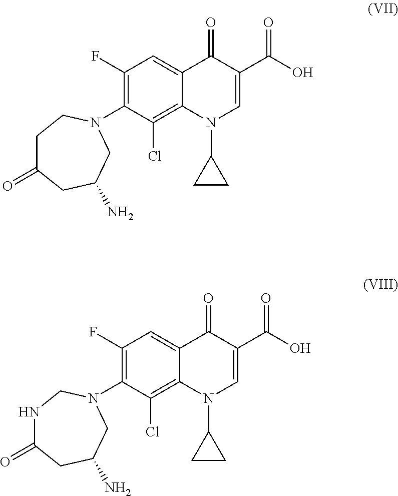

In still another aspect, a composition of the present invention comprises an enantiomer of one of the compounds having Formula I, II, or III.

In still another aspect, a composition of the present invention comprises a mixture of enantiomers of one of the compounds having Formula I, II, or III.

In yet another aspect, a composition of the present invention has a pH in the range from about 3.5 to about 5.5 (or, alternatively, from about 3.5 to 5, or from about 4 to 5, or from about 4 to 5.5), or from about 10.5 to about 12 (or, alternatively, from about 10.5 to 11, or from about 11 to 12).

In a further aspect, a composition of the present invention further comprises an additional anti-infective medicament that is selected from the group consisting of an antibacterial agent other than the quinolones having Formulae I, II, III, IV, V, VI, VII, and VIII; an antifungal agent; an antiviral agent; an antiprotozoal agent; and combinations thereof.

In another aspect, the additional antibacterial agent other than the quinolones having Formulae I, II, III, IV, V, VI, VII, and VIII comprises another quinolone.

In still another aspect, said another quinolone is selected from the group consisting of cinoxacin, ciprofloxacin, clinafloxacin, difloxacin, enoxacin, fleroxacin, gatifloxacin, grepafloxacin, levofloxacin, lomefloxacin, miloxacin, moxifloxacin, nadifloxacin, norfloxacin, ofloxacin, pazufloxacin, pefloxacin, sitafloxacin, sparfloxacin, temafloxacin, tosufloxacin, trovafloxacin, mixtures thereof, and combinations thereof.

In yet another aspect, said another quinolone has a MIC₉₀ value for a Gram-negative bacterium that is lower than that of the fluoroquinolone having Formula I, II, III, IV, V, VI, VII, or VIII for the same bacterium, said fluoroquinolone being included in the composition. MIC₉₀ is the minimum concentration of the active compound required to inhibit ninety percent of the growth of a specified pathogen, in μg/ml.

Non-limiting examples of antibacterial agents other than the quinolones having antibacterial agent other than the quinolones having Formulae I, II, III, IV, V, VI, VII, and VIII include biologically-derived antibacterial agents such as aminoglycosides (e.g., amikacin, apramycin, arbekacin, bambermycins, butirosin, dibekacin, dihydrostreptomycin, fortimicin(s), gentamicin, isepamicin, kanamycin, micronomicin, neomycin, neomycin undecylenate, netilmicin, paromomycin, ribostamycin, sisomicin, spectinomycin, streptomycin, tobramycin, trospectomycin), amphenicols (e.g., azidamfenicol, chloramphenicol, florfenicol, thiamphenicol), ansamycins (e.g., rifamide, rifampin, rifamycin sv, rifapentine, rifaximin), β-lactams (e.g., carbacephems (e.g., loracarbef), carbapenems (e.g., biapenem, imipenem, meropenem, panipenem), cephalosporins (e.g., cefaclor, cefadroxil, cefamandole, cefatrizine, cefazedone, cefazolin, cefcapene pivoxil, cefclidin, cefdinir, cefditoren, cefepime, cefetamet, cefixime, cefinenoxime, cefodizime, cefonicid, cefoperazone, ceforanide, cefotaxime, cefotiam, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephradine, pivcefalexin), cephamycins (e.g., cefbuperazone, cefinetazole, cefininox, cefotetan, cefoxitin), monobactams (e.g., aztreonam, carumonam, tigemonam), oxacephems, flomoxef, moxalactam), penicillins (e.g., amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin G benethamine, penicillin G benzathine, penicillin G benzhydrylamine, penicillin G calcium, penicillin G hydrabamine, penicillin G potassium, penicillin G procaine, penicillin N, penicillin O, penicillin V, penicillin V benzathine, penicillin V hydrabamine, penimepicycline, phenethicillin potassium, piperacillin, pivampicillin, propicillin, quinacillin, sulbenicillin, sultamicillin, talampicillin, temocillin, ticarcillin), ritipenem, lincosamides (e.g., clindamycin, lincomycin), macrolides (e.g., azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, miokamycin, oleandomycin, primycin, rokitamycin, rosaramicin, roxithromycin, spiramycin, troleandomycin), polypeptides (e.g., amphomycin, bacitracin, capreomycin, colistin, enduracidin, enviomycin, fusafungine, gramicidin s, gramicidin(s), mikamycin, polymyxin, pristinamycin, ristocetin, teicoplanin, thiostrepton, tuberactinomycin, tyrocidine, tyrothricin, vancomycin, viomycin, virginiamycin, zinc bacitracin), tetracyclines (e.g., apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, tetracycline), cycloserine, mupirocin, and tuberin.

Non-limiting examples of synthetic antibacterial agents include 2,4-diaminopyrimidines (e.g., brodimoprim, tetroxoprim, trimethoprim), nitrofurans (e.g., furaltadone, furazolium chloride, nifuradene, nifuratel, nifurfoline, nifurpirinol, nifurprazine, nifurtoinol, nitrofurantoin), quinolones and analogs (e.g., cinoxacin, ciprofloxacin, clinafloxacin, difloxacin, enoxacin, fleroxacin, flumequine, gatifloxacin, grepafloxacin, levofloxacin, lomefloxacin, miloxacin, moxifloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin, sulfonamides (e.g., acetyl sulfamethoxypyrazine, benzylsulfamide, chloramines B, chloramines T, dichloramine T, n²-formylsulfisomidine, n⁴-β-D-glucosylsulfanilamide, mafenide, 4′-(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidochrysoidine, sulfamoxole, sulfanilamide, 4-sulfanilamidosalicylic acid, n⁴-sulfanilylsulfanilamide, sulfanilylurea, N-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole) sulfones (e.g., acedapsone, acediasulfone, acetosulfone sodium, dapsone, diathymosulfone, glucosulfone sodium, solasulfone, succisulfone, sulfanilic acid, p-sulfanilylbenzylamine, sulfoxone sodium, thiazolsulfone), clofoctol, hexedine, methenamine, methenamine anhydromethylene citrate, methenamine hippurate, methenamine mandelate, methenamine sulfosalicylate, nitroxoline, taurolidine, and xibomol. In one embodiment, a composition of the present invention comprises an anti-infective agent selected from the group consisting of cinoxacin, ciprofloxacin, clinafloxacin, difloxacin, enoxacin, fleroxacin, flumequine, gatifloxacin, grepafloxacin, levofloxacin, lomefloxacin, miloxacin, moxifloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, and trovafloxacin.

Non-limiting examples of antiviral agents include rifampin, ribavirin, pleconaryl, cidofovir, acyclovir, pencyclovir, gancyclovir, valacyclovir, famciclovir, foscarnet, vidarabine, amantadine, zanamivir, oseltamivir, resquimod, adenosine arabinoside, cytosine arabinoside, antiproteases, PEGylated interferon (Pegasys™), anti HIV proteases (e.g. lopinivir, saquinivir, amprenavir, HIV fusion inhibitors, nucleotide HIV RT inhibitors (e.g., AZT, lamivudine, abacavir), non-nucleotide HIV RT inhibitors, doconosol, interferons, butylated hydroxytoluene (BHT), and hypericin.

Non-limiting examples of biologically-derived antifungal agents include polyenes (e.g., amphotericin B, candicidin, dermostatin, filipin, fungichromin, hachimycin, hamycin, lucensomycin, mepartricin, natamycin, nystatin, pecilocin, perimycin), azaserine, griseofulvin, oligomycins, neomycin undecylenate, pyrroInitrin, siccanin, tubercidin, and viridin.

Non-limiting examples of synthetic antifungal agents include allylamines (e.g., butenafine, naftifine, terbinafine), imidazoles (e.g., bifonazole, butoconazole, chlordantoin, chlormidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbamates (e.g., tolciclate, tolindate, tolnaftate), triazoles (e.g., fluconazole, itraconazole, saperconazole, terconazole), acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, and zinc propionate.

Non-limiting examples of antiprotozoal agents include polymycin B sulfate, bacitracin zinc, neomycine sulfate (e.g., Neosporin®), imidazoles (e.g., clotrimazole, miconazole, ketoconazole), aromatic diamidines (e.g., propamidine isethionate, brolene), polyhexamethylene biguanide (“PHMB”), chlorhexidine, pyrimethamine (Daraprim®), sulfadiazine, folinic acid (leucovorin), clindamycin, and trimethoprim-sulfamethoxazole.

The amount of a fluoroquinolone compound having Formula I, II, III, IV, V, VI, VII, or VIII that is incorporated into a composition of the present invention should be within a range sufficient to permit ready application of the composition to the affected tissue area in an amount which will deliver the desired amount of compound to the desired treatment site and to provide the desired therapeutic effect. In some embodiments of the present invention, compositions comprise a fluoroquinolone having Formula I, II, III, IV, V, VI, VII, or VIII in a concentration in a range from about 0.0001 percent to 10 percent by weight (or alternatively, from about 0.001 percent to about 5 percent, or from about 0.01 percent to about 5 percent, or from about 0.01 percent to about 2 percent, or from about 0.01 percent to about 1 percent, or from about 0.01 percent to about 0.7 percent, or from about 0.01 percent to about 0.5 percent, by weight).

In a still another aspect, said concentration is greater than about 0.05 percent by weight (or, alternatively, greater than about 0.1, or 1 percent by weight) of the total composition.

In still another aspect, said concentration is in the range from about 0.05 percent to about 2 percent by weight (or, alternatively, from about 0.05 percent to about 1 percent by weight) of the total composition.

In another aspect, the concentration of said additional anti-infective medicament in a composition of the present invention can be in the range from about 0.0001 percent to 10 percent by weight (or alternatively, from about 0.001 percent to about 5 percent, or from about 0.01 percent to about 5 percent, or from about 0.01 percent to about 2 percent, or from about 0.01 percent to about 1 percent, or from about 0.01 percent to about 0.7 percent, or from about 0.01 percent to about 0.5 percent, or from about 0.05 percent to about 0.5 percent, or from about 0.05 percent to about 1 percent, by weight).

In still another aspect, a composition of the present invention further comprises a pharmaceutically acceptable carrier.

A fluoroquinolone compound and, optionally, an additional anti-infective medicament that are disclosed herein can be formulated into a pharmaceutical composition for topical, oral, or systemic administration for the treatment or control of an infection. In an embodiment, such infection comprises an infection of an eye, an ear, a portion of a respiratory system, or a combination thereof. Such a composition comprises a fluoroquinolone compound having Formula I, II, III, IV, V, VI, VII, or VIII, an optionally additional anti-infective medicament, and a pharmaceutically acceptable carrier for the administration, which carrier can be determined by a person having skill in the art of pharmaceutical formulation for the applications disclosed above. For example, various pharmaceutically acceptable carriers known in the art can be used to formulate a solution, suspension, dispersion, ointment, gel, capsule, or tablet. In one embodiment, the fluoroquinolone compound is dissolved in the carrier of these dosage forms. A fluoroquinolone compound having Formula I, II, III, IV, V, VI, VII, or VIII disclosed herein is particularly suitable for a treatment or control of infections of an eye, an ear, a portion of a respiratory system, or a combination thereof. In a preferred embodiment, the composition is an aqueous solution.

In one embodiment, such infections are caused by at least one bacterium. When the additional anti-infective agent is an antifungal agent, an antiviral agent, or an antiprotozoal agent, a composition of the present invention can be used to treat or control mixed types of microorganisms comprising at least a bacterium and a fungus, at least a bacterium and a virus, or at least a bacterium and a protozoa, respectively. In another embodiment, such a fluoroquinolone and an additional anti-infective medicament or agent are formulated into a solution, ointment, suspension, dispersion, or gel.

In one embodiment, a topical composition of the present invention comprises an aqueous solution or suspension. Typically, purified or deionized water is used. The pH of the composition is adjusted by adding any physiologically acceptable pH adjusting acids, bases, or buffers to within the range from about 3.5 to about 5.5 (or, alternatively, from about 3.5 to 5, or from about 4 to 5, or from about 4 to 5.5), or from about 10.5 to about 12 (or, alternatively, from about 10.5 to 11, or from about 11 to 12).

Non-limiting examples of acids include acetic, boric, citric, lactic, phosphoric, hydrochloric, and the like, and examples of bases include sodium hydroxide, sodium phosphate, sodium borate, sodium citrate, sodium acetate, sodium lactate, tromethamine, THAM (trishydroxymethylamino-methane), and the like. Salts and buffers include citrate/dextrose, sodium bicarbonate, ammonium chloride and mixtures of the aforementioned acids and bases. pH buffers are introduced into the composition to maintain a stable pH or to improve product tolerance by the user. Biological buffers for various pHs are available, for example, from Sigma-Aldrich. A composition of the present invention can have a viscosity in the range from about 1 to about 100,000 centipoise (“cp”) or mPa·s (or alternatively, from about 5 to about 50,000, or from about 10 to about 20,000, or from about 10 to about 10,000, or from about 10 to about 1,000, or from about 100 to about 10,000, or from about 100 to about 20,000, or from about 100 to about 50,000 or from about 500 to about 10,000, or from about 500 to about 20,000 cp).

In another embodiment, a topical composition of the present invention comprises an ointment, emulsion or cream (such as oil-in-water emulsion), or gel.

Ointments generally are prepared using either (1) an oleaginous base; i.e., one consisting of fixed oils or hydrocarbons, such as white petrolatum or mineral oil, or (2) an absorbent base; i.e., one consisting of an anhydrous substance or substances which can absorb water, for example anhydrous lanolin. Customarily, following formation of the base, whether oleaginous or absorbent, the active ingredient (compound) is added to an amount affording the desired concentration.

Creams are oil/water emulsions. They consist of an oil phase (internal phase), comprising typically fixed oils, hydrocarbons, and the like, such as waxes, petrolatum, mineral oil, and the like, and an aqueous phase (continuous phase), comprising water and any water-soluble substances, such as added salts. The two phases are stabilized by use of an emulsifying agent, for example, a surface active agent, such as sodium lauryl sulfate, hydrophilic colloids, such as acacia colloidal clays, veegum, and the like. Upon formation of the emulsion, the active ingredient (compound) customarily is added in an amount to achieve the desired concentration.

Gels comprise a base selected from an oleaginous base, water, or an emulsion-suspension base. To the base is added a gelling agent which forms a matrix in the base, increasing its viscosity. Examples of gelling agents are hydroxypropyl cellulose, acrylic acid polymers, and the like. Customarily, the active ingredient (compound) is added to the formulation at the desired concentration at a point preceding addition of the gelling agent.

Moreover, a topical composition of the present invention can contain one or more of the following: preservatives, surfactants, adjuvants including additional medicaments, antioxidants, tonicity adjusters, viscosity modifiers, and the like.

Preservatives may be used to inhibit microbial contamination of the product when it is dispensed in single or multidose containers, and can include: quaternary ammonium derivatives, (benzalkonium chloride, benzylammonium chloride, cetylmethyl ammonium bromide, cetylpyridinium chloride), benzethonium chloride, organomercury compounds (Thimerosal, phenylmercury acetate, phenylmercury nitrate), methyl and propyl p-hydroxy-benzoates, betaphenylethyl alcohol, benzyl alcohol, phenylethyl alcohol, phenoxyethanol, and mixtures thereof. These compounds are used at effective concentrations, typically from about 0.005 percent to about 5 percent (by weight), depending on the preservative or preservatives selected. The amount of the preservative used should be enough so that the solution is physically stable; i.e., a precipitate is not formed, and antibacterially effective.

The solubility of the components, including a fluoroquinolone having Formula I, II, III, IV, V, VI, VII, or VIII of the present compositions may be enhanced by a surfactant or other appropriate co-solvent in the composition or solubility enhancing agents like cyclodextrins such as hydroxypropyl, hydroxyethyl, glucosyl, maltosyl and maltotriosyl derivatives of α-, β-, and γ-cyclodextrin. In one embodiment, the composition comprises 0.1 percent to 20 percent hydroxypropyl-β-cyclodextrin; alternatively, 1 percent to 15 percent (or 2 percent to 10 percent) hydroxypropyl-β-cyclodextrin. Co-solvents include polysorbates (for example, polysorbate 20, 60, and 80), polyoxyethylene/polyoxypropylene surfactants (e.g., Pluronic® F68, F84, F127, and P103), cyclodextrin, fatty-acid triglycerides, glycerol, polyethylene glycol, other solubility agents such as octoxynol 40 and tyloxapol, or other agents known to those skilled in the art and mixtures thereof. The amount of solubility enhancer used will depend on the amount of fluoroquinolone in the composition, with more solubility enhancer used for greater amounts of fluoroquinlones. Typically, solubility enhancers are employed at a level of from 0.01 percent to about 20 percent (alternatively, from about 0.1 percent to about 10 percent, from about 0.1 percent to about 5 percent, or from about 0.1 percent to about 2 percent) by weight depending on the ingredient.

The use of viscosity enhancing agents to provide the compositions of the invention with viscosities greater than the viscosity of simple aqueous solutions may be desirable to increase absorption of the active compounds by the target tissues or to increase the retention time in the ear. Such viscosity enhancing agents include, for example, polyvinyl alcohol, polyvinyl pyrrolidone, methyl cellulose, hydroxypropylmethyl cellulose, hydroxyethyl cellulose, carboxymethyl cellulose, hydroxypropyl cellulose or other agents know to those skilled in the art. Such agents are typically employed at a level of from about 0.01 percent to about 10 percent (alternatively, from about 0.1 percent to about 5 percent, or from about 0.1 percent to about 2 percent) by weight.

Suitable surfactants include polyvinyl pyrolidone, polyvinyl alcohol, polyethylene glycol, ethylene glycol, and propylene glycol. Other surfactants are polysorbates (such as polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamers (synthetic block polymers of ethylene oxide and propylene oxide, such as those commonly known by their trade names of Pluronic®; e.g., Pluronic® F127 or Pluronic® F108)), or poloxamines (synthetic block polymers of ethylene oxide and propylene oxide attached to ethylene diamine, such as those commonly known by their trade names of Tetronic®; e.g., Tetronic® 1508 or Tetronic® 908, etc., other nonionic surfactants such as Brij®, Myrj®, and long chain fatty alcohols (i.e., oleyl alcohol, stearyl alcohol, myristyl alcohol, docosohexanoyl alcohol, etc.) with carbon chains having about 12 or more carbon atoms (e.g., such as from about 12 to about 24 carbon atoms). The surfactant helps a topical formulation to spread on the surface of narrow passages.

It is often that an infection is followed by inflammation. Therefore, in another aspect, a composition of the present invention further comprises an anti-inflammatory agent. Anti-inflammatory agents include the well-known glucocorticosteroids and the non-steroidal anti-inflammatory drugs (“NSAIDs”).

Non-limiting examples of the glucocorticosteroids are: 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortarnate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, their physiologically acceptable salts, combinations thereof, and mixtures thereof.

The preferred glucocorticoids for ocular use include dexamethasone, prednisolone, methasone, fluorometholone, loteprednol, medrysone, and rimesolone. The preferred glucocorticoids for otic use include dexamethasone, loteprednol, rimexolone, prednisolone, fluorometholone, hydrocortisone, and their derivatives. The preferred glucocorticoids for nasal use include mometasone, fluticasone, beclomethasone, flunisolide, triamcinolone, budesonide, and their derivatives.

Non-limiting examples of the NSAIDs are: aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), ε-acetamidocaproic acid, S-(5′-adenosyl)-L-methionine, 3-amino-4-hydroxybutyric acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, their physiologically acceptable salts, combinations thereof, and mixtures thereof. In one embodiment, the NSAID is diclofenac, furbiprofen, or ketorolac.

Other non-steroidal anti-inflammatory agents include the cyclooxygenase type II selective inhibitors, such as celecoxib, and etodolac; platelet activating factor (“PAF”) antagonists, such as apafant, bepafant, minopafant, nupafant, and modipafant; phosphodiesterase (“PDE”) IV inhibitors, such as ariflo, torbafylline, rolipram, filaminast, piclamilast, cipamfylline, and roflumilast; inhibitors of cytokine production, such as inhibitors of the NF-κB transcription factor; or other anti-inflammatory agents known to those skilled in the art.

The concentrations of the anti-inflammatory agents contained in the compositions of the present invention will vary based on the agent or agents selected and the type of inflammation being treated. The concentrations will be sufficient to reduce inflammation in the targeted tissues following topical application of the compositions to those tissues. Such concentrations are typically in the range from about 0.0001 to about 3 percent by weight (or alternatively, from about 0.01 to about 2 percent, or from about 0.05 percent to about 1 percent, by weight).

Bacterial pathogens that have been isolated from cases of eye infection include gram-negative bacteria (such as Neisseria species, Moraxella species, Brucella species, Enterobacter species, Escherichia coli, Haemophilus influenzae, Klebsiella pneumoniae, and Pseudomonas aeruginosa), gram-positive bacteria (Staphylococcus aureus, Staphylococcus epidermidis, Streptococcus pneumoniae, Streptococcus pyogenes, Streptococcus mitis, Streptococcus faecalis, Bacillus subtilis, Clostridium tetani, Corynebacterium diphtheriae, Propionibacterium acnes, and Actinomyces israelii.

Bacterial pathogens that have been isolated from cases of ear infection include Pseudomonas aeruginosa, Staphylococcus aureus, Streptococcus pneumoniae, Streptococcus pyrogenes, Streptococcus faecalis, Haemophilus influenzae, Moraxella catarahalis, Escherichia coli, Proteus species, Klebsiella species, and Enterococcus species. Several of these species from the isolates have been found to be resistant to a number of antimicrobial drugs. For example, a published study of antimicrobial-resistant pathogens in middle-ear fluid of children with acute otitis media shows that thirty percent of the S. pneumoniae isolates were intermediately or fully resistant, and eight percent fully resistant, to penicillin; ten percent of the isolates were resistant to amoxicillin or amoxicillin-clavulanate. M. R. Jacobs et al., Antimicrobial Agents and Chemotherapy, Vol. 42, No. 3, 589 (1998). The same study shows that thirty percent of H. influenzae isolates produced β-lactamase, and thus, were expected to be resistant to penicillin.

Bacterial pathogens that have been isolated from cases of upper respiratory infection include Staphylococcus aureus, Staphylococcus epidermidis, Streptococcus pneumoniae, Streptococcus pyrogenes, Haemophilus influenzae, Peptostreptococcus species, and Bacteroides species. Some of these strains have been found to be resistant to commonly used antibiotics, as mentioned above.

Anti-bacterial activity of the compound having Formula IV was tested against several Gram-negative reference bacteria strains and compared to the anti-bacterial activity of three commercially available antibiotics (nadifloxacin, ofloxacin, and sparfloxacin). The results are shown in Table 2 as MIC₉₀ values (minimum concentration of the active compound required to inhibit ninety percent of the growth of a specified pathogen, in μg/ml).

TABLE 2
Comparison of In vitro Anti-bacterial Activity of Compound
Having Formula IV, Nadifloxacin, Ofloxacin, and Sparfloxacin
Against Some Gram-Negative Bacteria

| | MIC₉₀ (μg/ml) | | | |
|---|---|---|---|---|
| | Compound | | | |
| | Having | Nadi- | Oflox- | Spar- |
| Strain | Formula IV | floxacin | acin | floxacin |
| Escherichia coli | 0.1 | 0.2 | 0.05 | 0.012 |
| (O-1) | | | | |
| Klebsiella pneumoniae | 0.024 | 0.1 | 0.024 | 0.012 |
| (IFO 13541) | | | | |
| Salmonella typhimurium | 0.05 | 0.2 | 0.05 | 0.012 |
| (TD) | | | | |
| Shigella flexneri | 0.006 | 0.006 | 0.006 | 0.006 |
| (2b) | | | | |
| Enterobacter aerogenes | 0.39 | 1.56 | 0.2 | 0.1 |
| (IFO 13534) | | | | |
| Serratia marcenscens | 0.2 | 1.56 | 0.1 | 0.2 |
| (NHL) | | | | |
| Proteus mirabilis | 0.1 | 0.1 | 0.05 | 0.1 |
| (IFO 13300) | | | | |
| Proteus rettgeri | 0.39 | 0.39 | 0.2 | 0.2 |
| Acinetobacter calcoaceticus | 0.39 | 0.78 | 0.39 | 0.024 |
| (IFO 12552) | | | | |
| Pseudomonas aeruginosa | 1.56 | 3.13 | 1.56 | 0.78 |
| (IFO 13736) | | | | |
| Pseudomonas aeruginosa | 1.56 | 6.25 | 3.13 | 0.78 |
| (ATCC 27853) | | | | |

Anti-bacterial activity of the compound having Formula IV was tested against several Gram-positive reference bacteria strains and compared to the anti-bacterial activity of three commercially available antibiotics (nadifloxacin, ofloxacin, and sparfloxacin). The results are shown in Table 3 as MIC₉₀ values.

TABLE 3
Comparison of In vitro Anti-bacterial Activity of Compound
Having Formula IV, Nadifloxacin, Ofloxacin, and Sparfloxacin
Against Some Gram-Positive Bacteria

| | MIC₉₀ (μg/mL) | | | |
|---|---|---|---|---|
| | Compound | | | |
| | Having | Nadi- | Oflox- | Spar- |
| Strain | Formula IV | floxacin | acin | floxacin |
| Bacillis subtilis | 0.012 | 0.006 | 0.05 | 0.024 |
| (ATCC 6633) | | | | |
| Staphylococcus aureus | 0.05 | 0.05 | 0.39 | 0.1 |
| (ATCC 25933) | | | | |
| Staphylococcus aureus | 0.012 | 0.024 | 0.2 | 0.024 |
| (FDA 209P) | | | | |
| Staphylococcus aureus | 0.006 | 0.024 | 0.2 | 0.05 |
| (Smith) | | | | |
| Staphylococcus epidermidis | 0.05 | 0.1 | 0.78 | 0.2 |
| (ATCC 12228) | | | | |
| Sarcina lutea | 0.1 | 0.39 | 3.13 | 1.56 |
| (ATCC 9341) | | | | |
| Streptococcus faecalis | 0.1 | 0.78 | 1.56 | 0.78 |
| (IFO 12964) | | | | |
| Streptococcus faecalis | 0.2 | 0.78 | 1.56 | 0.39 |
| (ATCC 29212) | | | | |
| Streptococcus pyogenes | 0.1 | 0.78 | 0.78 | 0.39 |
| (Cook) | | | | |
| Streptococcus pyogenes | 0.1 | 0.1 | 0.39 | 0.2 |
| (IID 698) | | | | |
| Streptococcus pneumoniae | 0.1 | 0.78 | 1.56 | 0.39 |
| (IID 553) | | | | |
| Streptococcus pneniae | 0.2 | 0.78 | 1.56 | 0.39 |
| (IID 554) | | | | |

Anti-bacterial activity of the compound having Formula IV was tested against some methicillin-resistant Staphylococcus aureus bacteria strains and compared to the anti-bacterial activity of three commercially available antibiotics (nadifloxacin, ofloxacin, and sparfloxacin). The results are shown in Table 4 as MIC₉₀ values.

TABLE 4
Comparison of In vitro Anti-bacterial Activity of Compound Having
Formula IV, Nadifloxacin, Ofloxacin, and Sparfloxacin Against Some
Methicillin-Resistant Staphylococcus aureus Isolates

| | MIC₉₀ (μg/mL) | | | |
|---|---|---|---|---|
| | Compound | | | |
| | Having | Nadi- | Oflox- | Spar- |
| Strain | Formula IV | floxacin | acin | floxacin |
| Staphylococcus aureus | 0.012 | 0.006 | 0.05 | 0.024 |
| (ATCC 33591) | | | | |
| Staphylococcus aureus | 0.05 | 0.05 | 0.39 | 0.1 |
| (ATCC 33592) | | | | |
| Staphylococcus aureus | 0.012 | 0.024 | 0.2 | 0.024 |
| (ATCC 33593) | | | | |
| Staphylococcus aureus | 0.006 | 0.024 | 0.2 | 0.05 |
| (No. 395) | | | | |
| Staphylococcus aureus | 0.05 | 0.1 | 0.78 | 0.2 |
| (No. 415) | | | | |
| Staphylococcus aureus | 0.1 | 0.39 | 3.13 | 1.56 |
| (No. 419) | | | | |
| Staphylococcus aureus | 0.1 | 0.78 | 1.56 | 0.78 |
| (No. 420) | | | | |
| Staphylococcus aureus | 0.2 | 0.78 | 1.56 | 0.39 |
| (No. 421) | | | | |

Anti-bacterial activity of the compound having Formula IV was tested against selected anaerobic reference bacteria and compared to the anti-bacterial activity of three commercially available antibiotics (nadifloxacin, ofloxacin, and sparfloxacin). The results are shown in Table 5 as MIC₉₀ values.

TABLE 5
Comparison of In vitro Anti-bacterial Activity of Compound
Having Formula IV, Nadifloxacin, Ofloxacin, and Sparfloxacin
Against Some Common Anaerobic Bacteria

| | MIC₉₀ (μg/mL) | | | |
|---|---|---|---|---|
| | Compound | | | |
| | Having | Nadi- | Oflox- | Spar- |
| Strain | Formula IV | floxacin | acin | floxacin |
| Clostridium perfringens | 0.2 | 0.2 | 0.78 | 0.39 |
| (KZ 210) | | | | |
| Peptostreptococcus micros | 0.2 | 0.39 | 0.78 | 0.78 |
| (GIFU 7824) | | | | |
| Peptostreptococcus magnum | 0.05 | 0.2 | 0.39 | 0.1 |
| (GAI 0664) | | | | |
| Propionibacterium acnes | 0.2 | 0.2 | 0.78 | 0.39 |
| (GAI 5419) | | | | |
| Propionibacterium acnes | 0.2 | 0.2 | 0.78 | 0.39 |
| (ATCC 6919) | | | | |
| Propionibacterium acnes | 0.39 | 0.78 | 1.56 | 0.78 |
| (ATCC 11828) | | | | |
| Bacteroides fragillis | 0.1 | 1.56 | 1.56 | 0.78 |
| (GAI 0675) | | | | |
| Bacteroides thetaiotaomicron | 0.2 | 1.56 | 3.13 | 0.78 |
| (GAI 0659) | | | | |
| Bacteroides vulgatus | 0.1 | 0.78 | 3.13 | 0.78 |
| (GAI 0673) | | | | |
| Veillonella parvula | 0.1 | 0.78 | 0.39 | 0.2 |
| (GAI 5602) | | | | |

Anti-bacterial activity of the compound having Formula IV was tested against some ophthalmologic clinical bacteria isolates and compared to the anti-bacterial activity of three commercially available antibiotics (nadifloxacin, ofloxacin, and ciprofloxacin). As disclosed above, most of these bacteria strains are also relevant to infections of the ear and upper respiratory tract. The results are shown in Table 6 as MIC₉₀ values.

TABLE 6
Comparison of In vitro Anti-bacterial Activity of Compound
Having Formula IV, Nadifloxacin, Ofloxacin, and Ciprofloxacin
Against Some Clinical Bacteria Isolates

| | MIC₉₀ (μg/mL) | | | |
|---|---|---|---|---|
| | Compound | | | |
| | Having | Nor- | Oflox- | Cipro- |
| Strain | Formula IV | floxacin | acin | floxacin |
| Gram-negative | | | | |
| Moraxella species | 0.13 | 0.25 | 0.13 | ≦0.06 |
| Pseudomonas aeruginosa | 4 | 4 | 4 | 0.5 |
| Haemophilus influenzae | ≦0.06 | ≦0.06 | ≦0.06 | ≦0.06 |
| Gram-positive | | | | |
| Staphylococcus aureus | 1 | >128 | 32 | 64 |
| Coagulase-negative | 0.5 | 128 | 8 | 16 |
| Staphylococcus | | | | |
| Streptococcus pneumoniae | 0.13 | 8 | 2 | 1 |
| Corynebacterium species | 2 | 16 | 32 | 8 |
| Propionibacterium acnes | 0.25 | 4 | 1 | 0.5 |
| Antibiotic-resistant | | | | |
| Organisms | | | | |
| Ofloxacin-resistant | 16 | 128 | 64 | 32 |
| Enterobacteriaceae | | | | |
| Ofloxacin-resistant | 8 | >128 | >128 | >128 |
| Staphylococcus aureus | | | | |
| Gentamycin-resistant | 4 | 128 | 128 | 128 |
| Staphylococcus aureus | | | | |
| Gentamycin-resistant | 32 | 128 | 64 | 32 |
| Pseudomonas aeruginosa | | | | |
| Penicillin-resistant | 0.13 | 8 | 2 | 1 |
| Streptococcus pneumoniae | | | | |

The results show that the compound having Formula IV is effective against bacteria, including some antibiotic-resistant strains, which have been found in cases of infection of the eye, ear, or upper respiratory tract. Although the applicants do not wish to be bound by any particular theory, they believe that the unique moieties on the fluoroquinolones disclosed herein provide their advantageous antibacterial property and are effective for the treatment, reduction, amelioration, or prevention of infection of the ear, including otitis externa and otitis media, and infection of the upper respiratory tract, including sinusitis, nasopharyngitis, and oropharyngitis.

### Solubility and Stability of Solution Containing Compound Having Formula IV

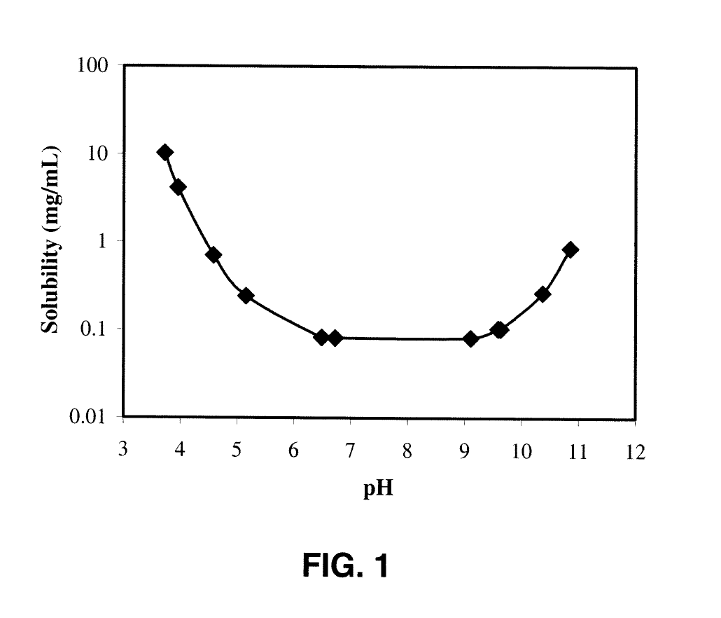

The solubility of the compound having Formula IV in USP water is shown in FIG. 1. Compositions of the present invention having pH in the ranges from about 3.5 to about 5.5 or from about 10.5 to about 12 can contain a high concentration of the compound and, thus, can deliver an effective amount of the drug to the affected site.

The stability of the compound having Formula IV in solution is shown in Table 7, as the remaining amount (expressed as the percentage of the initial amount) of the compound after selected times. The compound is stable in the pH range of most interest for the present invention.

TABLE 7
Stability of the Compound Having Formula
IV (Initial Concentration of 100 μg/ml)

| | | | |
|---|---|---|---|
| Time (week) | 0 | 2 | 4 |

| | | | | |
|---|---|---|---|---|
| pH | 3.5 | 100 | 99.0 | 98.8 |
| | 4.0 | 100 | 98.5 | 98.4 |
| | 4.5 | 100 | 98.4 | 98.4 |
| | 5.0 | 100 | 98.2 | 98.8 |
| | 5.5 | 100 | 96.8 | 96.8 |
| | 6.0 | 100 | 96.3 | 98.0 |
| | 6.5 | 100 | 95.3 | 95.6 |

The following examples are provided to further illustrate non-limiting compositions of the present invention, and methods of preparing such composition, for the treatment, reduction, amelioration, or prevention of infections.

### Example 1

### Antibacterial Solution

| Ingredient | Amount (percent by weight) |
|---|---|
| Compound having Formula IV | 0.3 |
| Hydroxypropylmethyl cellulose | 0.5 |
| (“HPMC”), medium viscosity (“MV”) | |
| Benzakonium chloride (“BAK”) | 0.01 |
| Pluronic ® F127 | 0.1 |
| EDTA | 0.1 |
| NaCl | 0.25 |
| Succinate buffer (0.05M, pH = 4.0) | q.s. to 100 |

An appropriate amount of succinate buffer is placed in a sterilized stainless steel jacketed vessel equipped with a stirring mechanism, at a temperature in the range from 50 to 60° C. The resulting buffer solution is heated to 61 to 75° C. At a temperature of about 66° C., an appropriate amount of BAK is added to the buffer solution while mixing three to ten minutes. At a temperature of 75° C., an appropriate amount of the compound having Formula IV is added to the contents of the vessel over a period of three to fifteen minutes while mixing continues. EDTA and NaCl are then added to the mixture while mixing continues for five to fifteen more minutes at 75° C. The resulting mixture is then sterilized, for example by autoclaving at a temperature of about 121° C. for about 30 minutes. The sterilized mixture is cooled to 25 to 30° C. The final composition is packaged in appropriate containers.

### Example 2

### Antibacterial Solution

A procedure similar to that of Example 1 is used to produce this solution.

| Ingredient | Amount (percent by weight) |
|---|---|
| Compound having Formula IV | 0.5 |
| Carboxymethyl cellulose (CMC) MV | 1 |
| Benzakonium chloride (“BAK”) | 0.01 |
| Pluronic ® F68 | 0.1 |
| EDTA | 0.1 |
| Sodium chloride | 0.25 |
| Acetate buffer (0.05M, pH = 4.5) | q.s. to 100 |

### Example 3

### Antibacterial and Anti-Inflammatory Solution

A procedure similar to that of Example 1 is used to produce this solution having the following composition.

| Ingredient | Amount (percent by weight) |
|---|---|
| Compound having Formula IV | 0.4 |
| Neomycin | 0.25 |
| Dexamethasone | 0.1 |
| Hydroxypropylmethyl cellulose | 0.5 |
| (“HPMC”) | |
| Alexidine | 0.01 |
| Brij ® surfactant | 0.1 |
| EDTA | 0.1 |
| Citrate buffer (0.02M sodium citrate, | q.s. to 100 |
| pH = 5) | |

### Example 4

### Antibacterial, Anti-Fungal, and Anti-Inflammatory Solution

A procedure similar to that of Example 1 is used to produce this solution having the following composition.

| Ingredient | Amount (percent by weight) |
|---|---|
| Compound 8 of Table 1 | 0.3 |
| Chlormidazole | 0.2 |
| Colecoxib | 0.15 |
| Propylene glycol | 0.5 |
| Alexidine | 0.01 |
| Tyloxapol | 0.1 |
| EDTA | 0.1 |
| Citrate buffer (0.02M sodium citrate, pH = | q.s. to 100 |
| 5) | |

### Example 5

### Antibacterial, Antiviral, and Anti-Inflammatory Suspension

A procedure similar to that of Example 1 is used to produce this solution having the following composition.

| Ingredient | Amount (percent by weight) |
|---|---|
| Compound having Formula VI | 0.3 |
| Rifampin | 0.25 |
| Triamcinolone, micronized USP | 0.2 |
| Hydroxyethyl cellulose | 0.25 |
| BAK | 0.01 |
| Tyloxapol | 0.05 |
| EDTA | 0.01 |
| NaCl | 0.3 |
| Na₂SO₄ | 1.2 |
| Sulfuric acid and/or NaOH | q.s. for pH adjustment to 5.5 |
| Citrate buffer (0.02M sodium citrate, | q.s. to 100 |
| pH = 5) | |

### Example 6

### Antibacterial and Anti-Inflammatory Emulsion

A modification of the procedure of Example 1 is used to produce this emulsion having the composition shown in the table immediately below.

Polysorbate 60 (Tween 60) is added to water in a first sterilized stainless steel jacketed vessel, equipped with a stirring mechanism, at a temperature of 50° C. to 60° C. in amounts corresponding the proportions shown in the table below. The resulting aqueous solution is heated to 61° C. to 75° C. At a temperature of 66° C., benzyl alcohol (a preservative) is added to the aqueous solution while mixing three to ten minutes. At a temperature of 75° C., appropriate amounts of the compound having Formula IV and loteprednol etabonate are added to the first vessel. An appropriate amount of Mygliol oil is charged into a second sterilized vessel, also equipped with a stirring mechanism, over a period of three to five minutes while stirring continues. Sorbitan monostearate and cetyl stearyl alcohol are added to the oil. The resulting oil mixture is heated to a temperature in the range from 62° C. to 75° C. The oil mixture is then added with vigorous mixing to the aqueous solution in the first vessel at a temperature of 66° C. over a period of three to five minutes. Sodium sulfate and sulfuric acid and/or sodium hydroxide are added to the mixture to adjust pH to 5.5. The resulting composition is cooled to 35° C. to 45° C. and homogenized by mixing with a high shear emulsifier or running through a homogenizer. The composition is then sterilized, for example by autoclaving at a temperature of about 120° C. The sterilized composition is further cooled to 25° C. to 30° C. The final composition is packaged in appropriate containers.

| Ingredient | Amount (percent by weight) |
|---|---|
| Compound having Formula IV | 0.1 |
| Loteprednol etabonate | 0.2 |
| Polysorbate 60 | 1 |
| Sorbitan monostearate (an emulsifier) | 1.5 |
| Cetyl stearyl alcohol (an emulsion | 1.5 |
| stabilizer) | |
| Benzyl alcohol | 0.5 |
| Miglyol oil | 14.5 |
| Na₂SO₄ | 1.2 |
| Sulfuric acid and/or NaOH | q.s. for pH adjustment to 5 |
| Purified water | q.s. to 100 |

Typically, the oil used in an emulsion is a non-irritating emollient oil. Illustrative but non-limiting examples thereof include a mineral oil, vegetable oil, and a reformed vegetable oil of known composition. More specific but non-limiting examples of the oil can be selected from the group consisting of peanut oil, sesame seed oil, cottonseed oil, and a medium chain (C₆ to C₁₂) triglycerides (e.g., Miglyol Neutral Oils 810, 812, 818, 829, 840, etc., available from Huls America Inc.). Typical emulsifiers employed can be selected from the group consisting of sorbitan monostearate and Tween 60 (also known as Polysorbate 60). Preferably, the emulsifiers are nonionic. The emulsifiers can be employed in an amount of 1.5 to 6.5 percent by weight of the composition, and preferably, 3 to 5 percent by weight of the composition. The hydrophobic phase of the emulsion can be in an amount of 15 to 25 percent by weight of the composition, and preferably, 18 to 22 percent by weight of the composition.

### Example 7

### Antibacterial, Antifungal, and Anti-Inflammatory Emulsion

A procedure similar to that of Example 6 is used to produce this emulsion having the following composition.

| Ingredient | Amount (percent by weight) |
|---|---|
| Compound 13 of Table 1 | 0.2 |
| Butoconazole | 0.2 |
| Triamcinolone, micronized USP | 0.2 |
| Polysorbate 60 | 1 |
| Sorbitan monostearate | 1.5 |
| Cetyl stearyl alcohol | 1.5 |
| Benzyl alcohol | 0.5 |
| Miglyol oil | 14.5 |
| Na₂SO₄ | 1.2 |
| Sulfuric acid and/or NaOH | q.s. for pH adjustment to 5 |
| Purified water | q.s. to 100 |

### Example 8

### Antibacterial Ointment

A procedure similar to that of Example 1 is used to produce this solution having the following composition.

| Ingredient | Amount (percent by weight) |
|---|---|
| Compound having Formula VI | 0.3 |
| Apramycin | 0.2 |
| White petrolatum USP | 50 |
| Propylene glycol | 5 |
| Glycerin | 5 |
| Tween ® 20 | 2 |
| Vitamin E | 1 |
| BAK | 0.1 |
| Mineral oil | q.s. to 100 |

### Example 9

### Antibacterial and Anti-Inflammatory Ointment

A procedure similar to that of Example 1 is used to produce this solution having the following composition.

| Ingredient | Amount (percent by weight) |
|---|---|
| Compound having Formula IV | 0.3 |
| Gatifloxacin | 0.1 |
| Dexamethasone | 0.15 |
| White petrolatum USP | 50 |
| Propylene glycol | 5 |
| Glycerin | 5 |
| Tween ® 20 | 2 |
| Vitamin E | 1 |
| Vitamin D | 0.5 |
| BAK | 0.1 |
| Mineral oil | q.s. to 100 |

### Example 10

### Antibacterial and Antiviral Solution

A procedure similar to that of Example 1 is used to produce this solution.

| Ingredient | Amount (percent by weight) |
|---|---|
| Compound having Formula IV | 0.35 |
| Mannitol | 4.5 |
| Adenine arabinoside | 0.3 |
| Benzakonium chloride (“BAK”) | 0.005 |
| Polysorbate 80 | 0.1 |
| EDTA | 0.05 |
| Sodium acetate | 0.03 |
| Acetic acid | 0.04 |
| Purified water | q.s. to 100 |

### Example 11

### Antibacterial and Antifungal Solution

A procedure similar to that of Example 1 is used to produce this solution.

| Ingredient | Amount (percent by weight) |
|---|---|
| Compound having Formula IV | 0.2 |
| Compound having Formula VI | 0.2 |
| Amphotericin B | 0.3 |
| Mannitol | 4.5 |
| Benzakonium chloride (“BAK”) | 0.005 |
| Polysorbate 80 | 0.1 |
| EDTA | 0.05 |
| Sodium acetate | 0.03 |
| Acetic acid | 0.04 |
| Purified water | q.s. to 100 |

### Example 12

### Antibacterial Emulsion

The procedure of Example 6 is used to produce this emulsion having the composition shown in the table immediately below.

| Ingredient | Amount (percent by weight) |
|---|---|
| Compound having Formula IV | 0.2 |
| Moxifloxacin | 0.2 |
| Polysorbate 60 | 1 |
| Sorbitan monostearate (an emulsifier) | 1.5 |
| Cetyl stearyl alcohol (an emulsion | 1.5 |
| stabilizer) | |
| Benzyl alcohol | 0.5 |
| Miglyol oil | 14.5 |
| Na₂SO₄ | 1.2 |
| Sulfuric acid and/or NaOH | q.s. for pH adjustment to 5 |
| Purified water | q.s. to 100 |

### Example 13

### Antibacterial Solution Containing Compound Having Formula IV

A procedure similar to that of Example 1 is used to produce this solution.

| Ingredient | Amount (percent by weight) |
|---|---|
| Compound having Formula IV | 0.6 |
| CMC MV | 1 |
| Pluronic ® F68 | 0.1 |
| EDTA | 0.1 |
| Sodium chloride | 0.25 |
| Potassium chloride | 0.1 |
| Formate buffer (0.05M, pH = 4) | q.s. to 100 |

### Example 14

### Antibacterial Solution Containing Compound Having Formula IV

A procedure similar to that of Example 1 is used to produce this solution.

| Ingredient | Amount (percent by weight) |
|---|---|
| Compound having Formula IV | 0.2 |
| Povidone | 3 |
| Triton 100 | 0.1 |
| EDTA | 0.1 |
| Benzalkonium chloride | 0.01 |
| Sodium chloride | 0.25 |
| Sorbitol | 3 |
| Phosphate buffer (0.05M, pH = 5) | q.s. to 100 |

### Example 15

### Antibacterial Solution Containing Compound Having Formula IV

A procedure similar to that of Example 1 is used to produce this solution.

| Ingredient | Amount (percent by weight) |
|---|---|
| Compound having Formula IV | 0.2 |
| CMC MV | 1 |
| Benzakonium chloride (“BAK”) | 0.01 |
| Pluronic ® F127 | 0.1 |
| EDTA | 0.1 |
| Sodium chloride | 0.15 |
| Mannitol | 2.5 |
| Phosphate buffer (0.03M) and citrate | q.s. to 100 |
| buffer (0.02M), pH = 4 | |

### Example 16

### Antibacterial Solution Containing Compound Having Formula IV and Anti-Inflammatory Drug

A procedure similar to that of Example 1 is used to produce this solution.

| Ingredient | Amount (percent by weight) |
|---|---|
| Compound having Formula IV | 0.3 |
| Loteprednol etabonate | 0.1 |
| CMC MV | 1 |
| Pluronic ® F127 | 0.2 |
| EDTA | 0.1 |
| Sodium chloride | 0.1 |
| Citrate buffer (0.05M, pH = 4) | q.s. to 100 |

### Example 17

### Antibacterial Composition Comprising Compound Having Formula IV and Ciprofloxacin

Compositions having various combinations of concentrations of compound having Formula IV and cirprofloxacin were tested systematically against S. aureus, P. aeruginosa, and E. coli. The MICs (in μg/ml) of each drug alone and in combination are shown in Table 8.

TABLE 8
MICs of Compound Having Formula IV and
Ciprofloxacin Alone and in Combination

| | Compound | | Compound | Cipro- |
|---|---|---|---|---|
| | Having | Cipro- | Having | floxacin |
| | Formula IV | floxacin | Formula IV in | in Com- |
| Organism | Alone | Alone | Combination | bination |
| S. aureus | 0.03 | 0.5 | 0.015 | 0.25 |
| (ATCC 29213) | | | 0.0005-0.001 | 0.5 |
| P. aeruginosa | 4 | 0.5 | 1 | 0.25 |
| (ATCC 27853) | | | 1-2 | 0.012 |
| | | | 2 | 0.06 |
| E. coli | 0.12 | 0.015-0.030 | ≦0.03 | 0.015 |
| (ATCC 25922) | | | 0.015-0.06 | 0.008 |
| | | | 0.03-0.06 | 0.004 |
| | | | 0.03-0.06 | 0.002 |
| | | | 0.015-0.03 | 0.008 |
| | | | 0.06 | 0.004 |

Data of Table 8 show that although ciprofloxacin alone is effective against S. aureus, combinations of the compound having Formula IV and ciprofloxacin can be more effective than either drug alone against P. aeruginosa and E. coli as MIC of at least one drug in the combination is less than one-half that of the same drug used alone.

The present invention also provides a method for treating or controlling infection of the eye, ear, or upper respiratory tract. In one aspect, such an infection is caused by mixed types of microorganisms including at least a bacterium. In another aspect, said at least a bacterium is one that is resistant to at least a commonly used antibiotic. In still another aspect, the method comprises administering one or more drops of a composition of the present invention to the ear canal, nasal cavity, or back of the throat of a subject who has indication of infection or whose risk of infection is indicated. A composition of the present invention can also be formulated into a spray, which can be administered into the otic or nasal cavity of such a subject.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A method for treating or controlling an infection in a subject, the method comprising administering to a subject in need thereof, a composition comprising a fluoroquinolone having Formula I or salts thereof, wherein the composition has a pH in a range from about 3.5 to about 5.5 or from about 10.5 to about 12, and the fluoroquinolone is present in an amount effective to treat or control said infection, 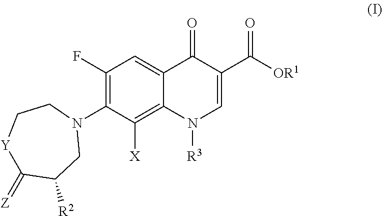
wherein R¹ is selected from the group consisting of hydrogen, unsubstituted lower alkyl groups, substituted lower alkyl groups, cycloalkyl groups, unsubstituted C₅-C₂₄ aryl groups, substituted C₅-C₂₄ aryl groups, unsubstituted C₅-C₂₄ heteroaryl groups, substituted C₅-C₂₄ heteroaryl groups, and groups that can be hydrolyzed in living bodies; R² is selected from the group consisting of hydrogen, unsubstituted amino group, and amino groups substituted with one or two lower alkyl groups; R³ is selected from the group consisting of hydrogen, unsubstituted lower alkyl groups, substituted lower alkyl groups, cycloalkyl groups, unsubstituted lower alkoxy groups, substituted lower alkoxy groups, unsubstituted C₅-C₂₄ aryl groups, substituted C₅-C₂₄ aryl groups, unsubstituted C₅-C₂₄ heteroaryl groups, substituted C₅-C₂₄ heteroaryl groups, unsubstituted C₅-C₂₄ aryloxy groups, substituted C₅-C₂₄ aryloxy groups, unsubstituted C₅-C₂₄ heteroaryloxy groups, substituted C₅-C₂₄ heteroaryloxy groups, and groups that can be hydrolyzed in living bodies; X is selected from the group consisting of halogen atoms; Y is selected from the group consisting of CH₂, O, S, SO, SO₂, and NR⁴, wherein R⁴ is selected from the group consisting of hydrogen, unsubstituted lower alkyl groups, substituted lower alkyl groups, and cycloalkyl groups; and Z is selected from the group consisting of oxygen and two hydrogen atoms; and wherein the composition is capable of inhibiting a growth or survival of at least a bacterium causing said infection.

2. The method of claim 1, wherein the composition is administered topically, orally, or systemically.

3. The method of claim 2, wherein the composition is administered topically.

4. The method of claim 3, wherein the infection is an infection of an eye, an ear, a portion of a respiratory system, or a combination thereof.

5. The method of claim 1, wherein a microorganism causing the infection is a bacterium that is resistant to an antibacterial agent.

6. The method of claim 1, wherein the composition further comprises a quinolone other than the fluoroquinolones having Formula I, and wherein the additional anti-infective agent is selected from the group consisting of antiviral agents, antifungal agents, antiprotozoal agents, and mixtures thereof.

7. A method for treating or controlling an infection in a subject, the method comprising administering to the subject in need thereof, a composition comprising: (a) a fluoroquinolone having Formula III or salts thereof; and (b) an additional anti-infective agent; wherein the composition has a pH in a range from about 3.5 to about 5.5 or from about 10.5 to about 12, and the fluoroquinolone has a concentration of from about 0.01 to about 1 percent by weight.

8. The method of claim 7, wherein the fluoroquinolone is a single enantiomer of the fluoroquinolone having Formula III.

9. The method of claim 7, wherein the composition is administered topically, orally, or systemically.

10. The method of claim 7, wherein the infection is an infection of an eye, an ear, a portion of a respiratory tract, or a combination thereof.
